(19) United States
(12) Patent Application Publication
(10) Pub. No.: US 2026/0083946 A1
(43) Pub. Date: Mar. 26, 2026

(54) IMPLANTABLE SENSORS AND ASSOCIATED SYSTEMS AND METHODS

(71) Applicant: Shifamed Holdings, LLC, Campbell, CA (US)

(72) Inventors: Brian Fahey, Menlo Park, CA (US); Amr Salahieh, Saratoga, CA (US); Marwan Berrada, Los Gatos, CA (US); Tom Saul, Portland, OR (US); Peter Andriola, Castro Valley, CA (US)

(21) Appl. No.: 19/407,301

(22) Filed: Dec. 3, 2025

Related U.S. Application Data
(63) Continuation of application No. 17/599,127.
(60) Provisional application No. 62/825,260, filed on Mar. 28, 2019.

(51) Int. Cl. A61M 27/00

(52) U.S. Cl. CPC A61M 27/002; A61M 2205/0266; A61M 2205/0294; A61M 2205/3324; A61M 2205/3344; A61M 2210/125

(57) ABSTRACT

The present technology relates to interatrial shunting systems and methods. In some embodiments, the present technology includes a system for shunting blood between a left atrium and a right atrium of a patient. The system can include a shunting element having a lumen extending therethrough. The lumen is configured to fluidly couple the left atrium and the right atrium when the shunting element is implanted in the patient. The system can also include a sensor configured to be implanted in the patient and operably coupled to the shunting element. An actuation element is coupled to the sensor. The actuation element is configured to be selectively energized and transmit a desired motion to the sensor to reduce the likelihood of tissue ingrowth/overgrowth and adhesion on at least a portion of the sensor.

### CROSS-REFERENCE TO RELATED APPLICATION(S)

This is a continuation of U.S. patent application Ser. No. 17/599,127, filed on Sep. 28, 2021, which is a 35 U.S. C. § 371 U.S. National Phase application of International Patent Application No. PCT/US2020/025509, filed on Mar. 27, 2020, which claims priority to U.S. Provisional Ser. No. 62/825,260 , filed on Mar. 28, 2019, the disclosures of which are incorporated herein by reference in their entireties.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present technology relates to implantable sensors and methods for making and using such devices.

### BACKGROUND

Implantable sensors can be used within the human body to measure a number of physiological parameters and enhance diagnostic and treatment paradigms. For example, sensors may be adapted to measure a patient's electrocardiogram (ECG), blood pressure in various locations, cardiac output, insulin levels, and other parameters of interest.

While some physiological sensors may be viable for external use (e.g., ECG), others may require an internal approach for accuracy, compliance, or other reasons. For example, central blood pressure measurements (e.g., pressure in the chambers of the heart, pulmonary vasculature, vena cava, etc.) may be estimated with external approaches, but due to a lack of consistency and accuracy with external techniques, invasive approaches are considered the medical gold-standard upon which condition management decisions are typically based. A challenge with invasive approaches is related to the time period over which parameters are measured. Some invasive measures are “spot checks” that capture parameters at single instant in time, for example via use of a catheter inserted into the vasculature. These acute measurements may or may not accurately reflect a patient's condition over a longer period of time in a variety of settings (e.g., at rest and during exertion), and may or may not be sufficient to manage a patient's condition chronically. Other invasive measures involve the use of implantable devices, which provide chronic measurement but are also associated with limitations-for example, implants are often permanent, can be associated with sensor drift over periods of time, or may be designed to operate passively in order to overcome challenges associated with battery-life.

### BRIEF DESCRIPTION OF THE DRAWINGS

FIG. 1 illustrates an implantable sensor apparatus configured in accordance with an embodiment of the present technology.

FIGS. 2A-2D illustrate an implantable sensor apparatus having dynamic stabilization features and configured in accordance with an embodiment of the present technology.

FIG. 3 is a schematic diagram of an electronics module configured as a patch worn on the skin of a patient in accordance with an embodiment of the present technology.

FIGS. 4A and 4B illustrates a second configuration of an implantable sensor system adapted to be completely implanted within the body of a patient and configured in accordance with an embodiment of the present technology.

FIGS. 5A-5D illustrate an implantable sensor apparatus configured in accordance with another embodiment of the present technology.

FIGS. 6A and 6B are schematic illustrations of an interatrial shunting system configured in accordance with an embodiment of the present technology.

### DETAILED DESCRIPTION

The present disclosure describes sensors (and methods of using and implanting them) that are configured for implantation into the body of a subject. The sensors may be configured for permanent implantation or temporary implantation within the subject. In some cases, temporary can be for an acute period (hours, 7 days, 30 days, 90 days, 6 months) or longer-term, but adapted to be removed. The period of implantation may be dictated by the underlying condition(s) being measured by the sensors, the design and use characteristics of the sensors and/or related equipment, or by a combination of these or other factors. Endothelialization typically initiates quickly after implantation, and tissue begins ingrowth on the implant within days. Within a relatively short time, tissue ingrowth can be significant enough that scarring will occur if the implant moves. Tissue ingrowth can also make removal of the implant clinically impossible or very risky. Thus, in some embodiments of the present technology, the implanted sensors may be operably coupled to stabilization features or actuation elements configured to periodically change a position or configuration of the sensors. These positional and/or configuration changes are expected to reduce the likelihood of tissue ingrowth/overgrowth and endothelialization on the corresponding sensors while implanted within the body of the subject.

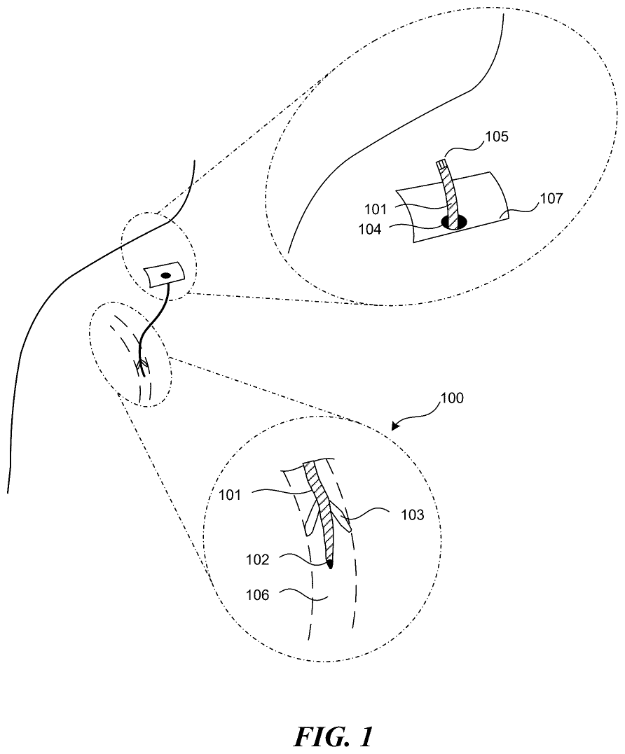

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-6B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

### Select Embodiments of Implantable Sensor Apparatuses

FIG. 1 illustrates an implantable sensor apparatus 100 (“apparatus 100”) configured in accordance with an embodiment of the present technology. As shown, the apparatus 100 comprises a thin elongated body 101 including a pressure transducer 102 at or near its distal terminating end, anchoring or stabilization features 103 proximate to the distal terminating end of the elongated body, secondary anchoring or anchoring-interface features 104 proximate to the proximal end of the elongated body, and electronic interface features 105 proximate to the proximal end that are adapted to convey information between the sensors on the sensor apparatus and electronic components (e.g., a microcontroller) that may be included on another apparatus that is part of the system. In other embodiments, the electronic components may be included as part of the implantable sensor apparatus and, accordingly, limited or no electronic interface features are necessary. In still further embodiments, the apparatus 100 may include a plurality of sensors adapted to measure a plurality of parameters, for example temperature, heart rate, ECG, blood pH, other chemical analyses, relative or absolute motion, flow of a fluid such as blood, lumen diameter, or other parameters. In the illustrated embodiment, the apparatus 100 is configured such that the distal end of the elongated body 101 is positioned within a blood vessel 106 and the proximal end of the elongated body remains at an interface point 107 which resides outside of the patient's body. As shown, the apparatus 100 is implanted in a location on the right side of the body. Those skilled in the art will understand, however, that the apparatus 100 is adapted to be positioned in a wide variety of different body location(s) in a patient.

In one embodiment, the elongated body 101 is configured to be very thin, for example without limitation, with a diameter similar to that of a guidewire (e.g. 0.14 inches, or 0.035 inches), so as to be capable of penetrating the wall of a blood vessel in a substantially atraumatic fashion. In other embodiments, the elongated body may have a more substantial thickness, for example a diameter on the order of 0.066-0.091 inches (5-7 Fr) and similar to that of a pacemaker lead, so as to avoid a “fishing wire” type of effect where the body may cut into tissues over time. Variations may involve even larger diameter elongated body configurations. In further embodiments, the elongated body may be of variable thickness, or be of adjustable thickness.

In some embodiments, the elongated body 101 is configured such that it may puncture a blood vessel wall without the need for any accompanying closure apparatus to be utilized while the body is indwelling or upon removal of the body. The elongated body 101 is preferably comprised of a flexible biocompatible material such as nitinol or annealed or partially-annealed stainless steel. The length of elongated body 101 will be adapted for the anticipated sensing location, for example measuring a parameter in a peripheral blood vessel or tissue region will generally require a shorter elongated body than measuring a parameter in a central blood vessel or an internally deep tissue region. In some embodiments, the elongated body 101 contains one or more internal lumens to allow for electrical signals, substances, materials, or other matter to move between the proximal and distal ends of the body. For example, an internal lumen may contain one or more electrical wires that allow for electrical power from a battery or other power source to reach a distal sensor and for communication of data from the sensor to electronics near the proximal end of the elongated body 101.

The apparatus 100 also includes one or more distal anchoring or stabilization features 103 that may be utilized to hold at least a portion of the elongated body 101 stable within a desired anatomical region. For example, stabilization feature(s) 103 may be adapted to hold the distal tip of the elongated body 101 proximate to a carotid bifurcation, in the aortic arch, in a section of a pulmonary artery, or within a segment of liver tissue. The design of the stabilization feature(s) 103 may vary depending upon the anatomy of the tissue region targeted for anchoring.

In some embodiments, the apparatus 100 is configured to be positioned within a blood vessel and adapted to record a measurement of local blood pressure. In some implementations of such embodiments, stabilization features 103 may be configured to be static following deployment. In other words, once deployed, stabilization features 103 will remain substantially in the same configuration until it is desired to remove the implanted apparatus 100. In some implementations, stabilization feature(s) 103 comprise one or more wire loops adapted to be foldable into tight contact with the elongated body 101 so as to present a slim apparatus profile during delivery/implantation, then fan radially outward as part of a deployment step (e.g., the removal of an introducer sheath). Wire loops may be constructed of a thin, shape-memory material such as nitinol and may be sized such that they exert a mild outward pressure against the interior walls of the blood vessel in which the implant sits. In other embodiments, the stabilization feature(s) 103 may comprise of a compliant, semi-compliant, or non-compliant balloon. In some implementations, the balloon may have one or more internal lumens so as to not substantially restrict blood flow when inflated. In further implementations, the stabilization feature(s) 103 may resemble a coil, such as a shape-memory nitinol coil that can be later withdrawn into the lumen of a delivery/removal device.

In some embodiments, the stabilization feature(s) 103 may be dynamic in nature and periodically change position or configuration. These positional and/or configuration changes may be in response to an algorithm stored on and executed by a system component (e.g., a microcontroller), in response to a user action, or in response to some other trigger that would be understood by those skilled in the art. One advantage of embodiments of the present technology is that implementing dynamic stabilization features is expected to reduce the likelihood of tissue ingrowth/overgrowth and endothelialization.

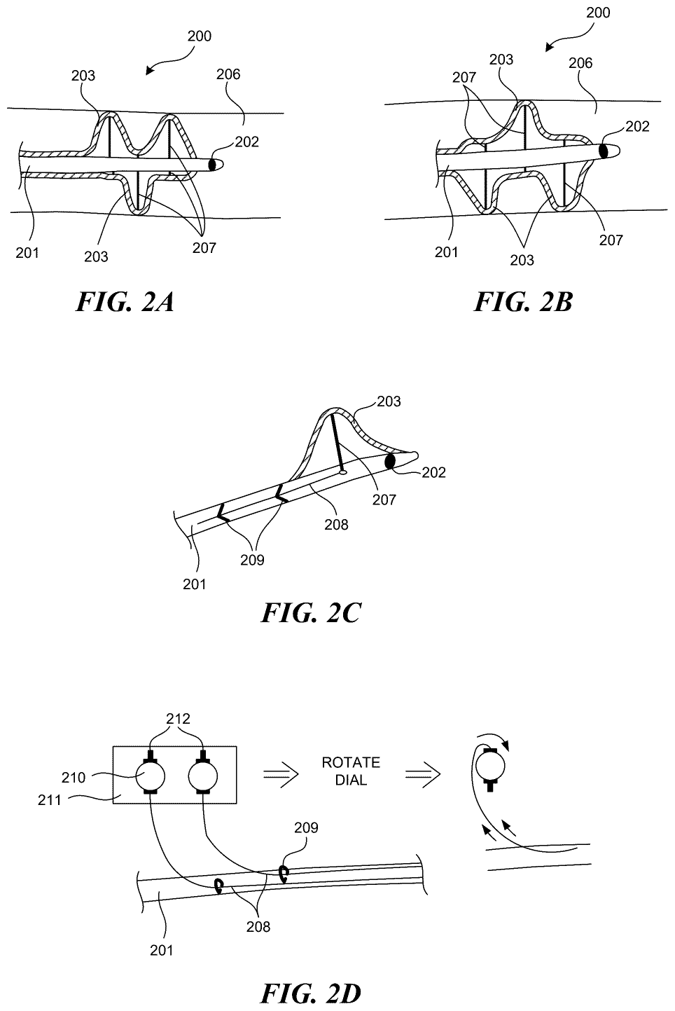

FIGS. 2A-2D illustrate an implantable sensor apparatus 200 (“apparatus 200”) utilizing dynamic stabilization features and configured in accordance with an embodiment of the present technology. A distal portion of a sensor implant is shown, with a physiological sensor 202 located proximate to the distal tip of elongated body 201, which is positioned within an anatomical lumen 206 of a patient or subject. FIG. 2A illustrates the apparatus 200 in an initial anchored state, with stabilization features 203 deployed in a first configuration or first state. Referring next to FIG. 2B, the stabilization features 203 adopt a second configuration or second in response to user input or an algorithmic trigger executed by software running on an electronic component of the system located at or near the proximal end of the elongated body (not shown). A number of suitable mechanisms for adjusting the configuration of stabilization features 203 are available and known to those skilled in the art. In the example shown in FIGS. 2A-2D, each stabilization feature 203 attaches to the elongated body 201 with a plurality of adjustable tethers 207. By manipulating the length of a tether 207, a portion of the stabilization feature 203 can be retracted close to the elongated body 201 or be extended outward toward the wall of the anatomical lumen 206. In some embodiments, the length of tethers 207, when fully-extended, are at least 10% larger than the radius of the lumen 206, allowing for unrestricted contact of the stabilization features 203 with the lumen wall.

FIG. 2C illustrates an embodiment in which the tethers 207 are adjusted in length by altering a position of tether control wires 208. In some embodiments, for example, tether control wires 208 are secured to the elongated body through semi-circular anchors 209 that exist periodically along the elongated body 201. In some embodiments, the inner diameter of the opening in anchors 209 is slightly larger than the outer diameter of control wires 208, thereby allowing for small translation of the wires 208 along the body 201 without the need for bearings or other motion facilitating components. FIG. 2D illustrates a mechanism by which a user may adjust the positions of control wires 208, and thus of tethers 207. In particular, control wires 208 branch off from the elongated body 201 near its proximal end and interface with dials 210 that reside on patch 211, which is adapted to sit on the exterior surface of a patient's body (e.g., resides on the skin-not shown). Control wires 208 enter through a first entry port in dial 210, travel through the center of the dial, and terminate in a fixed connection 212 located on the opposite side of the dial. With a dial 210 in a first position, the control wire(s) 208 are fully-advanced and, accordingly, the associated tether 207 is fully-extended. As a result, the portion of stabilization feature 203 proximate to the corresponding tether 207 extends outwardly from the elongated body 201 and contacts the wall of the lumen 206 (FIGS. 2A and 2B). Rotating dial 210 into a second position results in the control wire 208 becoming wrapped around the dial 210, which retracts the control wire 208 along the elongated body and thus pulls the associated tether 207 into a retracted position. This action results in the stabilization feature 203 associated with this tether 207 being retracted close to the elongated body and away from the wall of the body lumen. It should be understood that the aforementioned mechanisms for adjustment of a stabilization feature are provided by way of example only, and that multiple alternative configurations and mechanisms will be appreciated by those skilled in the art.

In some embodiments, the stabilization features 203 may be configured to serve additional functional purposes beyond anchoring and stabilizing the distal end (or other portions) of the elongated body. As one illustrative example, in some embodiments the stabilization features may themselves contain additional sensors that capture information about anatomical or physiological parameters. In some embodiments, for example, the stabilization features may be utilized to measure the diameter of the body lumen or otherwise estimate its size or shape. In other embodiments, the stabilization features may be utilized to estimate changes in the size or shape of the lumen, or to assess another relevant feature. In some implementations, accelerometers or other sensors may be integrated into stabilization features in order to assess lumen movements over time, for example how a blood vessel may change shape in response to pulsatile blood flow, or to measure how a blood vessel's shape changes are impacted by different measured pressures. In additional embodiments, mechanisms on either the stabilization features or on the elongated body itself measure the rate of flow that is traveling through a blood vessel. In some implementations, flow is measured directly. In other implementations, flow is estimated using formulaic calculations and other measured parameters such as pressure readings at one or more points along the elongated body, vessel diameter and/or size/shape, motion/acceleration, or other parameters.

Referring back to FIG. 1, the apparatus 100 may be configured such that it terminates in a region outside of the body. In such a configuration, for example, the elongated body 101 of a sensor apparatus that is adapted to measure the pressure within a blood vessel must exit the vasculature at a puncture point nearby the transition point to the body exterior. In embodiments, following the exit of the vasculature at the puncture point, a portion of the elongated body may tunnel through a subcutaneous region of tissue and exit the body. In some embodiments, an introducer sheath or other temporarily-implanted conduit may be employed to provide a path for the elongated body to traverse the tissue regions outside of the vasculature and reach the region external to the patient's body.

The elongated body 101 may include one or more secondary anchoring features 104 near its proximal end. In some embodiments, at least one secondary anchoring feature 104 is intended to act as a strain relief component at the puncture site where a sensor apparatus crosses into the patient's vasculature. For example, a circumferential strain relief may augment stability of the elongated body, may assist with sealing the vessel to prevent leakage, may restrict the motion of the elongated body in such a manner that it reduces the risk of injury to the vessel wall, may help prevent the spread of infection, and/or may serve other purposes. In some embodiments, at least one secondary anchoring feature exists at the exterior of the patient's body in order to secure the proximal end of the elongated body portion of the sensor apparatus and prevent undesired movement either into or out of the body.

In some embodiments, the apparatus 100 comprises electronic interface features 105 at or near the proximal terminus of the elongated body 101. Among potentially other uses, the electronic interface features 105 may enable communication between the sensor apparatus and other aspects of an implantable sensor system. For example, signals that characterize measured parameters may be relayed to electronics, processing components, or data transmission components that reside within a system component that resides outside of the body, for instance a patch on the patient's skin. Similarly, signals that control features of the sensor apparatus, e.g., signals instructing sensors to capture measurements or signals instructing stabilization features 103 to shift in position, may be sent from an external system component (not shown). In some implementations, the elongated body 101 terminates in a connector configuration, similar to connectors commonly used in the art. Such connectors allow multiple independent connections to interface simply with an external system component. In alternative implementations, the elongated body 101 may interface directly with an external system component without any connector/adaptor, i.e., similar to a wire being placed into a receptacle. In other embodiments, however, the implantable sensor apparatus lacks any electronic interface features.

In several embodiments, the apparatus 100 is a component of an implantable sensor system, with at least one additional system component being an electronics module configured to reside external to the patient. In some embodiments, for example, the electronics module is configured to function as a patch that adheres to the skin using medical-grade adhesives known to those skilled in the art (e.g., silicone or acrylic-based adhesives). The patch may be configured to remain on the skin over an extended period of time, e.g., 7 days, 30 days, 90 days, etc. As such, the patch may include other features, or interface with features, to enable an extended period of use. For example, the patch may include a waterproof or water resistant covering such that showering/bathing during the period of use does not impact the functionality of the electronics module or limit the period over which the patch may be worn.

In other embodiments, in lieu of an adhesive patch, the electronics module may be a slim-profile attachment intended to be worn by a patient, e.g., strapped to the patient using a configuration similar to an arm-band or watch. The elongated body of the sensor apparatus may be stabilized at its exit point from the skin, and the electronics module may be designed to be detachable. In some implementations, the electronics module is reusable and may be reattached after being decoupled from the sensor apparatus. In other implementations, the electronics module is intended to be disposable and, once it is removed from the body, may be discarded by the patient.

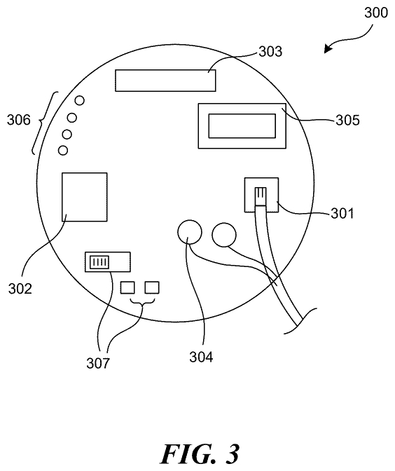

FIG. 3 is a schematic diagram of an electronics module 300 configured as a patch worn on the skin of a patient in accordance with an embodiment of the present technology. The electronics module 300 may include one or more of the following features: a sensor interface port 301, a CPU or similar control module 302, a wireless transmission antenna 303, a position adjustment feature 304, and a power supply feature 305. The electronics module 300 may also contain features such as indicators 306 that communicate information regarding the state of operation and various control features 307 that may enable a care provider or a user some control over system functionality. In some embodiments, the sensor interface port 301 is a magnetic or a snap connection that enables a non-technical user or patient, including those who may suffer from some degree of cognitive impairment, to easily establish or remove the connection between the sensor apparatus and the control module.

In some embodiments, the control module 302 is an electronic subsystem capable of being loaded with software and/or firmware that controls the basic functionality of the implantable sensor system. The control module 302, for example, may be based upon FPGA and/or microcontroller system architecture, or based upon other suitable electronic design architecture known to those skilled in the art. Updates to software/firmware running on control module 302 may be made via wireless communication protocols, e.g., via Bluetooth or Wi-Fi communication between the control module 307 and an external programmer (not shown). In other embodiments, however, the control module 302 may contain an adapter port to establish a direct wired connection to an external system for programming or other purposes. In still further embodiments, the control module 302 may not be reprogrammable following an initial programming and manufacturing process.

The electronics module 300 may also include a wireless transmission antenna 303 in electronic communication with the control module 302. The transmission antenna 303 preferably uses established communication protocols such as Bluetooth or Wi-Fi to facilitate communication with a broad network of existing commercially available wireless devices such as smartphones, home-based communication pods/smart speakers (e.g., Amazon Echo, Google Home), and others. In some implementations, the electronics module 300 includes limited on-board memory and data collected by a sensor are substantially streamed in real-time or semi-real-time away from the module to an external reader without significant on-board data storage. For example, a small data buffer may exist to store subsets of data in the event wireless communications are disabled or interrupted, but the module may lack memory to store a series of data representing sensed parameters over a broader period of time, e.g., over 30 minutes, over 60 minutes, over 24 hours. In other embodiments, however, the electronics module 300 may include significant on-board memory, e.g., flash memory, RAM, ROM, or another suitable form of memory known to those skilled in the art, which provides the option for both real-time streaming of data from a wireless antenna to an external reader or periodic and/or on-demand streaming of chunks of data that may have been captured during a prior time period and subsequently stored. In further embodiments, the electronics module 300 may not contain a wireless transmission antenna, and data communication to and from the module may occur via a direct connection, e.g., a USB, firewire, or lightning cable connection, or another suitable connection known to those skilled in the art. In additional embodiments, the electronics module 300 has no direct data communication capabilities, and instead data are read and/or written to a removable memory source, such as an SD card.

In some embodiments, the implantable sensor systems described herein are active systems powered via a battery. Accordingly, the electronics module 300 may include a power supply feature 305. The power supply feature 305 can include, for example, a thin-profile battery. Battery technologies are well-described in the prior art and will not be addressed here. The battery may be a primary cell battery or a rechargeable battery. The battery may be recharged, for example, using motion that occurs as the electronics module 300 is worn by an ambulatory subject (not shown). In other embodiments, the battery is recharged using an applied signal, such as a non-contact radiofrequency (RF) frequency signal supplied by another component in the environment of the patient. In further embodiments, the battery is not replaceable and the entire electronics module 300 must be replaced when the battery ceases supplying sufficient power to the system.

Referring to FIGS. 1-3 together, embodiments of the presently-disclosed implantable sensor systems may include features intended to reduce infection risk and/or features intended to limit the propagation of infection along the length of the elongated body of the sensor apparatus into central regions of the patient's body. In some embodiments, for example, all or some of the elongated body is coated with a silver material that is resistant to bacterial growth. In other embodiments, an ultraviolet (UV) light source is adapted to periodically illuminate infection-sensitive areas to prevent or limit the propagation of bacterial growth and/or biofilms. The ultraviolet light source may be activated at timed intervals and/or may be activated during activities thought to place the system at risk of infection, for example, after a patient or user touches or manipulates the electronics module for some purpose.

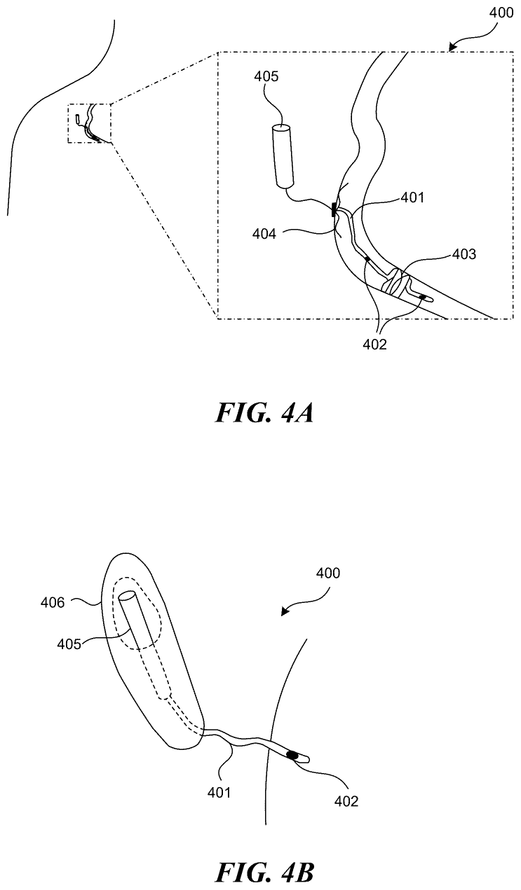

FIGS. 4A and 4B illustrate a second configuration of an implantable sensor system 400 (“system 400”) adapted to be completely implanted within the body of a patient and configured in accordance with an embodiment of the present technology. As there is no component of the system 400 emerging from the patient's body and exposed to sources of bacteria, the infection risk may be reduced or eliminated. As shown, an elongated body 401 is adapted to reside in the vasculature of the patient and contains or interfaces with one or more parameter-measuring sensors 402. Some embodiments may include at least one stabilization/anchoring feature 403 and/or one or more proximal anchoring features 404. As illustrated, the elongated body 401 exits the vasculature proximate to the location of a subdermal implant 405. The subdermal implant 405 is intended to be slim in profile and have an exterior portion constructed of a biocompatible material. The subdermal implant 405 may encapsulate various electronics as described above with reference to FIG. 3, e.g., a CPU/control module, battery, and/or wireless transmission antenna. The subdermal implant may be hermetically-sealed or otherwise similarly prepared such that its contents are protected from the body environment and vice-versa (i.e., chemicals from a malfunctioning battery cannot reach body tissues). A proximal end of the elongated body 401 can interface with the subdermal implant 405 via a connector mechanism or another docking-style interface. For clarity, many or all of the features described above in the context of an externally-worn/patch-type electronics module may also be adapted for use with embodiments that utilize an electronics module configured as a subdermal implant.

In some embodiments, the subdermal implant 405 is configured such that it may be injected into the body of the patient using a syringe, injection needle, or similar technique that enables a minimally-invasive procedure. Following deployment into a subdermal compartment, the subdermal implant 405 may be be interfaced with the elongated body 401 by medical personnel with or without establishing a subdermal tunnel between the point where the elongated body exits the vasculature and the location of the subdermal implant.

The subdermal implant 405 may be adapted to be removed from the body of the patient after a desired period of time, which may or may not be identical to the time period for which the entire sensor system is implanted. In such embodiments, tissue overgrowth of the implant may exacerbate challenges related to its removal. Accordingly, as best seen in FIG. 4B, some embodiments of the present technology include the use of a bioabsorbable shell 406 component to encapsulate the subdermal implant 405. The bioabsorbable shell 406 is preferably sized to be slightly larger than implant 405 and may be comprised of, for example, a sugar-based compound, a synthetic polymer such as polyglycolic acid (PGA) or polylactic acid (PLA), or another suitable material. The bioabsorbable shell 406 is intended to shield the implant 405 from tissue ingrowth, overgrowth, or adhesions to facilitate removal of the subdermal implant 405. The bioabsorbable shell 406 may not be retrieved during the removal of the implant 405 or the implantable sensor system as a whole. Instead it may optionally be left in place and slowly resorbed by the body. The period of resorption can be tailored, for example, such that the bioabsorbable shell 406 maintains sufficient structural integrity to isolate the subdermal implant 405 during the implantation period, then deteriorate shortly thereafter. In some embodiments, the subdermal implant 405 may be retrieved using a needle, syringe, tweezer apparatus, or another suitable tool that is adapted to enter the body transcutaneously, penetrate the shell, then secure and remove the implant. In some embodiments, an additional anchoring component may be utilized to secure the elongated body in place within or proximate to the bioabsorbable shell 406 until an additional step (e.g., the implantation of another implant, or the removal of the elongated body and entire system) can occur. In further embodiments, a bioabsorbable mesh, or alternative embodiments of bioabsorbable enclosures, may be utilized in lieu of the bioabsorbable shell 406.

As discussed above, it may be desirable to take active steps to limit any tissue overgrowth of the elongated body portion of an implantable sensor apparatus as it resides in the patient (either in the vasculature or a non-vasculature region) for a period of time. In addition to dynamically-positioned stabilization/anchoring features previously described, other approaches are possible and may be accompanied by meaningful advantages. In some embodiments, for example, the position of the elongated body may be altered periodically at timed intervals by forcing a pressurized stream of a fluid (e.g., saline) out of one or more exit ports located along the length of the body. In alternative embodiments, the implantable sensor apparatus may include a rigid or semi-rigid hypotube covering some length of the elongated body. The position of the hypotube may be altered, either automatically by the system or manually by a user, in such a way that the hypotube shears off and removes any tissue adhesions that have formed over portions or all of the elongated body. In further embodiments, the elongated body may periodically vibrate at frequencies and amplitudes intended to discourage adhesion to tissues including the walls of adjacent anatomical structures. Some embodiments may further include a specialized polymer or chemical coating to the elongated body and/or hypotube to further discourage or retard the onset of tissue overgrowth and adhesion.

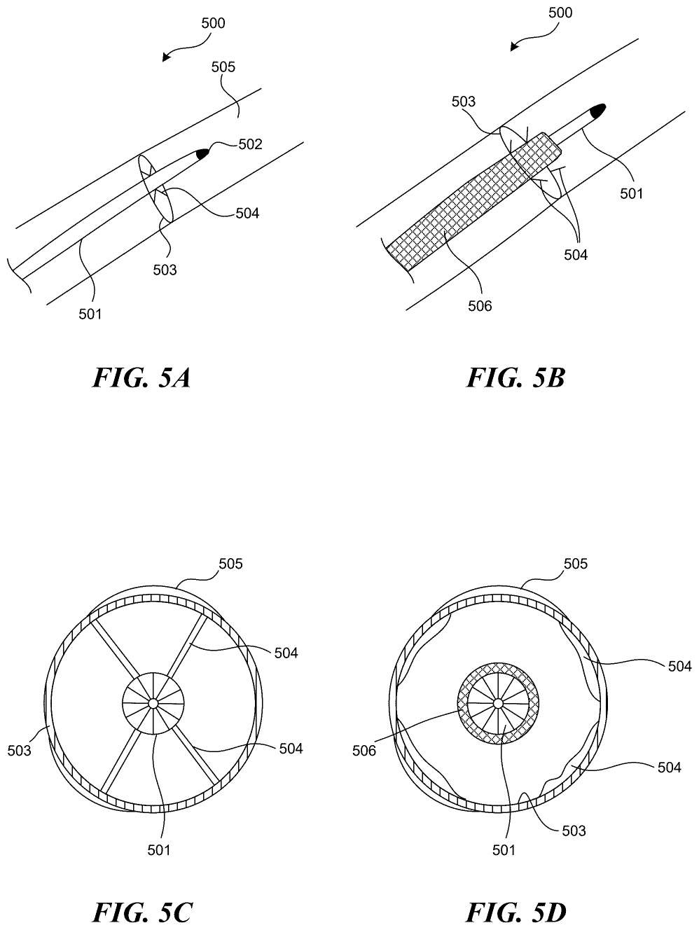

FIGS. 5A-5D illustrate an implantable sensor apparatus 500 (“apparatus 500”) configured in accordance with another embodiment of the present technology. This embodiment is directed to an alternative approach to preventing an elongated body 501 of the apparatus 500 from adhering to body tissues via overgrowth, ingrowth, or endothelialization (which may make the removal of the apparatus 500 challenging). FIG. 5A, for example, illustrates a distal portion of the apparatus 500 implanted within a vessel 505 of a patient. In the arrangement illustrated in FIG. 5A, the apparatus 500 is in a deployed configuration. The apparatus 500 comprises elongated body 501 and one or more deployable anchors or stabilization features 503 configured to hold the elongated body 501 and relevant parameter sensors 502 in place. The stabilization features 503 have a thin diameter and are preferably constructed of a material such as nitinol or stainless steel. The stabilization features 503 are connected to elongated body 501 via tethers 504. In some embodiments, the stabilization features 503 and/or the tethers 504 are bioabsorbable/biodegradable and are designed to be resorbed by the body over a period of time longer than the desired period of apparatus implantation. In some embodiments, the tethers 504 and/or the stabilization features 503 are designed to have shape memory properties that drive the mechanical behavior of the components. In practice, the stabilization features 503 (and, in some implementations, the tethers 504) are not designed to be retrieved from the body after deployment. As such, tissue overgrowth of the stabilization features may be of limited consequence.

FIG. 5B illustrates the apparatus 500 in a different arrangement during removal of the apparatus from the patient. During the removal process, a disengagement tool 506 may be utilized to separate the elongated body 501 from the stabilization features 503 and/or the tethers 504. In some embodiments, a protection filter may also be utilized in conjunction with removal to catch any materials and/or emboli dislodged during the disengagement process and prevent them from traveling downstream within the blood vessel. Following disengagement, the elongated body 501 is removed from the patient, leaving the stabilization feature(s) 503 behind. In some embodiments, the tethers 504 remain attached to the elongated body 501 and are removed from the body as well, with or without the aid of a sheath.

In alternative embodiments, the tethers 504 remain within the patient and are not removed. FIG. 5C, for example, is a cross-sectional view of the elongated body 501, stabilization features 503, and tethers 504 prior to disengagement, while FIG. 5D is a cross-sectional view of the components after disengagement. As best shown in FIG. 5D, disengagement occurs at the point where the tethers 504 meet the elongated body 501, and the shape memory properties of the tethers 504 cause them to move outward towards the vessel wall proximate to the stabilization feature(s) 503.

In embodiments of the present technology including implantable parameter sensors, the sensor apparatus is configured to assess blood analysis parameters using one or more blood or chemical sensors positioned at locations along the elongated body. The sensor apparatus, for example, may be configured to perform these blood assessments over time. In one embodiment, a small sample of blood is extracted and pulled into the elongated body at timed interval(s), and is sent through a lumen to a testing configuration located in the electronics module or elsewhere via a vacuum or pump mechanism. In other embodiments, test strips or sensors are built directly into the elongated body, and electrical indications of test results are transmitted to a communication source (e.g., via a wireless transmission antenna) located outside of the vessel, e.g., located on an electronics module positioned either on the patient's skin or inside of a subdermal implant. In additional embodiments, blood assay sensors are configured to have time-gated qualities that automatically or semi-automatically enable readings to happen at set intervals. In one implementation, blood assay test strips are embedded into the wall of the elongated body and covered with a bioabsorbable coating which, once eroded, exposes the test strip and triggers a measurement to be made. For example, test strip coatings may erode after 60 minutes, 7 days, 14 days, 30 days, some combination of these time periods, or at any arbitrary time period, in order to facilitate serial measurements of desired parameters. Once a measurement is obtained, the measured parameter or a characteristic indicative of the measured parameter is transmitted via the elongated body to an electronics module or to similar components of a system to enable the measured parameter to be displayed, recorded, transmitted, or otherwise interpreted in a medically-useful way.

Embodiments of the disclosed implantable sensor apparatuses that are adapted to reside in vasculature of a patient may be accompanied by a delivery system that is functionally similar to a Swan-Ganz catheter. More specifically, the sensor apparatus will dock or otherwise attach to or be contained within the delivery device, which may resemble a medical grade catheter known to those skilled in the art in terms of functionality and form factor. In general, delivery catheter access to the vasculature is obtained via a puncture to a peripheral vein, e.g., a femoral vein, a subclavian vein, an internal jugular vein, or another suitable vessel. The delivery catheter is then maneuvered to the desired location, either by an operator advancing the catheter forward manually, having the catheter “float” downstream by inflating a balloon and letting the force of directional blood flow direct the catheter to a desired location, via another technique, or through a combination of techniques.

Once the sensor apparatus has been positioned such that sensors/transducers are in the desired location, the delivery catheter unlatches, releases, withdraws, or otherwise decouples itself from the sensor apparatus in response to a user action, such as in response to a user manipulating a knob, dial, or other control feature on the proximal handpiece of the delivery catheter. In some implementations, the delivery system is designed, configured, and adapted such that a rapid exchange method of delivery is enabled. In some embodiments, the delivery catheter and/or sensor apparatus includes positional markers that are visible with imaging techniques commonly used in medical procedures, e.g., radiopaque markers that are visible on x-ray or fluoroscopy or acoustically-reflective markers that are visible under ultrasonic interrogation. Prior to or following the decoupling of the delivery catheter from the sensor apparatus, any stabilization or anchoring features that are integrated into the sensor apparatus may be deployed.

An alternative embodiment of an implantation system/delivery device involves a trocar-style method where an implantable sensor is delivered directly into a region of interest, for example directly into a muscle region, a fatty tissue region, an organ such as the liver, or into a central blood vessel. With or without the assistance of imaging guidance, e.g., ultrasound or fluoroscopy, a trocar or similar device is inserted transcutaneously into tissue proximate to the region of interest, with the cannula serving as an effective conduit to the deposition point. In an example scenario where the target site is a blood vessel (e.g., an inferior vena cava or pulmonary artery), the insertion cannula may be extended such that it abuts the wall of the vessel. A needle or another insertion device with a beveled edge may then be utilized to facilitate local introduction of the sensor apparatus directly into the region of interest. Such a delivery embodiment may be advantageous for an implantable sensor apparatus. For example, an elongated body inserted via a peripheral vessel but residing in the pulmonary artery must traverse several chambers of the heart and several heart valves to reach its targeted location, and over the course of months may present a risk for tissue injury due to repetitive contact and mechanical interactions with tissues that move many times per minute. Additionally, an elongated body that spans a large distance of vasculature may present an increased risk for partial or substantial endothelialization and tissue overgrowth/ingrowth.

In some alternative embodiments, one or more sensors of the sensing apparatus are not configured to be removed, or are not treated as a temporarily implantable device and are not removed. For example, referring to the embodiments described above with reference to FIGS. 4A and 4B, a distal portion of the elongated body containing at least one sensing device can be adapted to be left behind long-term (e.g., permanently), while other portions (e.g., a proximal portion) are removed after a shorter period of time. The implant portion maintained in the subject can be configured as a passive device that is activated with an external component of the system, and perform measurement(s) when activated. In these embodiments, during an initial period of time the sensing device can collect real-time or essentially real-time data without requiring user intervention. After a portion is removed, the sensing device can provide data when requested, e.g., via an interface with an external reader.

### Select Embodiments of Interatrial Shunting Systems

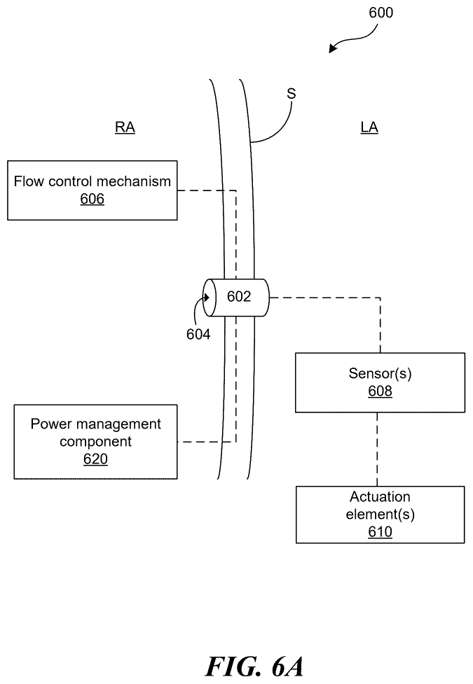

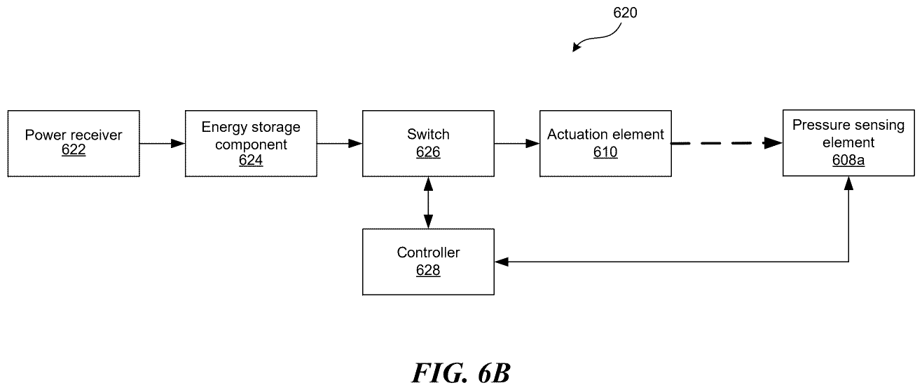

FIGS. 6A and 6B illustrate another embodiment of a system configured in accordance with an embodiment of the present technology. In particular, FIG. 6A is a schematic illustration of an interatrial shunting system 600 (“system 600”) implanted within a patient and adapted to selectively control a volume of blood flow between a right atrium RA and a left atrium LA of the patient. The system 600 includes, for example, a shunting element 602 defining a lumen 604 therethrough. When implanted in a septal wall S within a heart of a patient, the system 600 fluidly connects the left atrium LA and the right atrium RA via the lumen 604. The system 600 can also include various active electronic components carried by, operably coupled to, and/or otherwise associated with the shunting element 602, including one or more implantable sensors 608. The active electronic components, including the sensor(s) 608, are configured to perform various operations related to the shunting element 602. As described in further detail below, the system 600 may also include features to periodically alter the position of the sensor(s) 608 and other components of the system to limit or prevent tissue overgrowth and adhesion.

The system 600 comprises a flow control mechanism 606 configured to change a size, shape, and/or other characteristic of the shunting element 602 to selectively modulate the flow of fluid through the lumen 604. For example, the flow control mechanism 606 can be configured to selectively increase a diameter of the lumen 604 and/or selectively decrease a diameter of the lumen 604 in response to an input. In other embodiments, the flow control mechanism 606 is configured to otherwise affect a shape and/or geometry of the lumen 604. Accordingly, the flow control mechanism 606 can be operably coupled to the shunting element 602 and/or can be included within the shunting element 602 itself. In some embodiments, for example, the flow control mechanism 606 is part of the shunting element 602 and at least partially defines the lumen 604. In other embodiments, the flow control mechanism 606 is spaced apart from but operably coupled to the shunting element 602.

The sensor(s) 608 can be configured to measure one or more parameters of the system 600 (e.g., a characteristic or state of the shunting element 602 or lumen 604) and/or one or more physiological parameters of the patient (e.g., left atrial pressure, right atrial pressure, etc.). The sensor(s) 608 can be coupled to the shunting element 602 or can be positioned at a location within the heart spaced apart from the shunting element 602 (e.g., the left atrium LA, the right atrium RA, the septal wall S, etc.). For example, the system 600 can include a first sensor positionable within or proximate to the left atrium LA to measure left atrial pressure, and a second sensor positionable within or proximate to the right atrium RA to measure right atrial pressure. Examples of sensor(s) 608 suitable for use with the embodiments herein include, but are not limited to, pressure sensors, impedance sensors, accelerometers, force/strain sensors, proximity sensors, distance sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. In some embodiments, the system 600 includes multiple different types of sensors, such as at least two, three, four, five, or more different sensors and, as noted previously, the sensors may be positioned at a variety of different locations within the patient.

As discussed above, during operation it may be desirable to take active steps to limit any tissue overgrowth/adhesion of the sensor(s) 608 and/or other components of the system 600 while implanted in the patient. In some embodiments, for example, the system 600 may be configured to periodically actuate/move one or more of the sensors 608 within the patient. Such small movements of the implanted sensor(s) are expected to help prevent/inhibit adhesion of the sensor(s) to tissue, such as the walls of adjacent anatomical structures.

The sensor(s) 608, for example, may be operably coupled to one or more actuation elements 610 (shown schematically) or other suitable mechanisms to help facilitate the desired actuation/motion while the sensors are implanted in the patient. The individual actuation elements 610, for example, may comprise elements mechanically linked with or otherwise in communication with corresponding sensors 608. As described in greater detail below, actuation elements 610 may be energized periodically to transmit a desired motion to the sensors, thereby altering the position of the sensors.

The system 600 can also include and/or be operably coupled to a power management component 620 for powering operation of the various components, including the flow control mechanism 606, sensor(s) 608, and/or actuation element(s) 610. The power management component 620 can be configured to receive energy from an energy source positioned external and/or internal to a patient's body. The power management component 620 may also include one or more sources for providing any suitable combination of chargeable and non-rechargeable energy.

FIG. 6B, for example, is a schematic illustration of further details regarding the power management system 620 of FIG. 6A. In the illustrated embodiment, the power management system 620 includes a power receiver 622 operably coupled to an energy storage component 624, a switch 626, and a controller 628. The power receiver 622, energy storage component 624, switch 626, and controller 628 may be components already used for other power management functions in the system 600 (e.g., the flow control mechanism 606), or may be separate components used specifically for actuating/moving one or more sensors within the patient to prevent/limit any tissue overgrowth or adhesion.

The power receiver 622 may comprise, for example, a piezoelectric element configured to harvest energy from intentional or incidental bodily motion, pulsatile cardiac tissue motion, pulsatile hydraulic pressure variation in blood, and/or pulsatile Venturi effect pressure variations due to varying blood flow through the shunt. In additional embodiments, the power receiver 622 may also comprise other means of harvesting electromagnetic energy from ambient fields such as, for example, a radio receiver circuit capturing energy from cellular communications, WiFi, Bluetooth, WLAN (wireless local area network), WPAN (wireless personal area network), and/or WBAN (wireless body area network). In some embodiments, the power receiver 622 may also be coupled to a rectifier and boost converter (not shown) to deliver power to the energy storage component 624.

The power management system 620 further comprises a switch 626 operably coupled to actuation element 610 and adapted to selectively energize the actuation element 610. Energizing the actuation element 610 transmits a desired motion to a corresponding sensor, such as pressure sensing element 608a. The actuation element 610 may comprise, for example, an independent element mechanically linked to the pressure sensing element 608a. In one embodiment, the actuation element 610 comprises a shape memory element. The shape memory portion can include a shape memory metal or alloy such as nitinol, a shape memory polymer, a pH-based shape memory material, or any other suitable material configured to move or otherwise adjust in response to an input. For example, the actuation member 610 can include a nitinol element configured to change shape in response to applied heat that raises the nitinol element's temperature above the material transformation temperature. Such changes to the actuation member 610 accordingly result in movement/adjustment of the corresponding sensor to which the actuation member is attached (e.g., pressure sensing element 608a).

In other embodiments, the actuation element 610 may have other suitable configurations or features. For example, the actuation element 610 may comprise a bi-metallic strip or a piezo-electric bimorph that is mechanically linked to pressure sensing element 608a. The bi-metallic strip or a piezo-electric bimorph is configured to move/actuate the pressure sensing element 608a when energized by electrical power from energy storage component 624.

In another embodiment, the actuation element 610 may be integrally formed with or an internal component of pressure sensing element 608a. In one particular arrangement, for example, the pressure sensing element 608a comprises a capacitive pressure sensor including a capacitor plate configured to deform under pressure. The plate may also be used to transduce pressure information based on a change of voltage V at a known amount of charge Q, and convert electrical energy to motion (i.e., for actuation of the pressure sensing element itself) by means of varying said voltage.

Controller 628 is operably coupled to pressure sensing element 608a and configured to provide additional feedback/communication between pressure sensing element 608a and the power management system 620. In some embodiments, for example, pressure sensing element 608a may communicate with controller 628 and provide information used to estimate the degree of tissue overgrowth and current sensor calibration. In one particular embodiment in which the pressure sensing element 608a is a deformable plate of a capacitor, changes to the mechanical resonant frequency of the component may be measured by applying a voltage pulse and measuring the mechanical resonant frequency and damping by observing the transient capacitance variation. Such information can then be evaluated to determine when (or if) actuation/movement of the pressure sensing element 608a is required to alleviate or inhibit problems associated with tissue overgrowth/adhesion.

### EXAMPLES

Several aspects of the present technology are set forth in the following examples:
1. A sensing device configured to be temporarily positioned in a subject, the sensing device comprising:
   at least one sensor;
   at least one changeable member,
   wherein the at least one changeable member is configured and adapted so that at least one of its position and configuration can be adjusted after being placed in a target anatomical location in order to reduce the likelihood of tissue ingrowth/overgrowth and endothelialization of at least a portion of the sensing device.
2. The sensing device of example 1 wherein the at least one changeable member is disposed in a distal region of the sensing device.
3. The sensing device example 1 wherein the at least one changeable member is carried by, directly or indirectly, an elongate body (e.g., a shaft) of the sensing device, optionally in a distal portion of the elongate body.
4. The sensing device of example 1 wherein the at least one sensor is supported by an elongate shaft.
5. The sensing device of example 4 wherein the at least one sensor comprises a pressure sensor.
6. The sensing device of example 4 wherein the sensor is disposed distal to at least one changeable member, and optionally distal to any/all changeable members.
7. The sensing device of example 1 wherein at least one changeable member is in operational communication with an actuator, and optionally configured to be disposed external to the subject.
8. The sensing device of example 7 wherein the actuator can be manually actuated (e.g., actuator on a handle) and/or automatically actuated (e.g., via software/algorithm(s)) to operatively communicate with at least one changeable member.
9. The sensing device of example 7 wherein at least one changeable member is coupled (directly or indirectly) to a tether, the tether being in operative communication with the actuator such that actuation of the actuator causes a change in the tether (e.g., put in tension) and thereby changes at least one of position and configuration of the at least one changeable member.
10.T he sensing device of example 1 wherein at least one changeable member is a stabilization member configured and adapted to interface with adjacent tissue to increase stabilization of a least a portion of the sensing device relative to the adjacent tissue.
11. The sensing device of example 1 wherein the at least one changeable member is configured and adapted such that at least one of its position and configuration can be adjusted repeatedly over time, optionally at a plurality of discrete times.
12. The sensing device of example 1 wherein a changeable member comprises a sensor, which is optionally the sensor or a second sensor (e.g., an accelerometer).
13. The sensing device of example 1 wherein a proximal region of the sensing device is configured and adapted to be disposed external to the subject.
14. The sensing device of example 1 wherein the sensing device includes an anchoring member configured to anchor the sensing device at a location where the sensing device exits the subject, optionally wherein the anchoring member is configured and adapted to act as a seal.
15. The sensing device of example 1 wherein a proximal region of the sensing device is configured and adapted to be disposed inside the subject.
16. The sensing device of example 1, further comprising an electronics module, optionally in a proximal region, and optionally at a proximal end.
17. The sensing device of example 1 wherein the electronics module includes at least one of a communication component, processing component, a storage device, and a power supply.
18. The sensing device of example 1 wherein the electronic module is positioned to be disposed and maintained inside the subject.
19. The sensing device of example 1 wherein the electronic module is positioned to be disposed and maintained outside the subject.
20. The sensing device of example 1, further comprising a wearable component adapted to be secured to the subject.
21. The sensing device of example 1, further comprising one or more members adapted to limit the propagation of infection (e.g., a coating resistant to bacterial growth).
22. A method of using the sensing device of any of examples 1-21 wherein the method includes changing (manually or automatically) at least one of a position and configuration of at least one changeable member after the sensing device has been positioned in a target region.
23. The method of example 22 wherein the changing occurs at least one day after a procedure to place at least a portion of the sensing device in the subject, and optionally repeating the changing step a plurality of additional times.
24. The method of example 23 wherein the method comprises positioning the sensor in a blood vessel.
25. The method of example 24 wherein the method comprises reconfiguring at least one stabilization member to engage tissue and increase the stability of at least a portion of the sensing device relative to the tissue.
26. A sensing device configured to be temporarily positioned in a subject and also positioned completely within the subject (i.e., within an external surface of the subject), the sensing device comprising:
   an elongate body configured to be placed in a blood vessel of the subject, the elongate body including at least one sensor; and
   a subdermal housing coupled to the elongate body, the subdermal housing configured and sized to be implanted and temporarily maintained outside of the blood vessel.
27. The sensing device of example 26 wherein the elongate body includes one or more stabilization members.

28. The sensing device of example 26 wherein the elongate body includes one or more changeable members that are configured to have at least one of position and configuration changed after implantation.
29. The sensing device of example 28, further comprising at least one proximal stabilization member configured to anchor the elongate body at a proximal location where the elongate body exists the blood vessel.
30. The sensing device of example 26 wherein the subdermal housing includes at least one of a processing component, power source, communication component, and storage device.
31. The sensing device of example 26 wherein the subdermal housing is hermetically sealed.
32. The sensing device of example 26 wherein the subdermal housing includes a bioabsorbable shell configured to resist tissue ingrowth, overgrowth, or adhesions to facilitate removal of the subdermal housing.
33. The sensing device of example 32 wherein the shell can be detached from a remainder of the housing so that the shell can remain in the subject after the housing is removed.
34. The sensing device of example 26 wherein the sensing device is configured to continuously sense information from the subject.
35. A sensing device configured to be temporarily positioned in a subject, the sensing device comprising:
   an elongate body configured to be placed in a blood vessel of the subject, the elongate body comprising a sensor; and
   at least one stabilization member configured to be deployed and interface tissue to increase the stability of at least a portion of the sensing device relative to the blood vessel,
   wherein the at least one stabilization member is configured to be controllably detached from at least a portion of the sensing device after the elongate body has been placed in the blood vessel.
36. The sensing device of example 35 wherein the at least one stabilization member is bioabsorbable over a period of time, the resorption providing the controllable detachment from the sensing device.
37. The sensing device of example 35, further comprising a separate detachment body configured to contact at least one portion of the sensing tool to controllably detach the at least one stabilization member from the sensing device.
38. The sensing device of example 37 wherein the separate detachment body comprises an elongate body.
39. The sensing device of example 38, further comprising at least one breakable connecting member coupled to a stabilization member.
40. A method of using a sensing device of any of examples 35-39 comprising positioning the elongate body and sensor in the blood vessel.
41. The method of example 40, further comprising detaching a stabilization member from at least a portion of the sensing device after the positioning device.
42. The method of example 41 wherein the detaching step occurs at least 1 day after the positioning step.
43. The sensing device of example 35, further comprising any suitable features of examples 1-21 and 26-34.
44. A system for shunting blood between a left atrium and a right atrium of a patient, the system comprising:
   a shunting element having a lumen extending therethrough, wherein the lumen is configured to fluidly couple the left atrium and the right atrium when the shunting element is implanted in the patient;
   a sensor configured to be implanted in the patient and operably coupled to the shunting element; and
   an actuation element coupled to the sensor, wherein the actuation element is configured to be selectively energized and transmit a desired motion to the sensor to reduce the likelihood of tissue ingrowth/overgrowth and endothelialization of at least a portion of the sensor.
45. The system of example 44, further comprising an energy storage component implanted in the patient and configured to store energy, wherein the energy stored in the energy storage component can be used to actuate the actuation element.
46. The system of example 44 wherein the actuation element comprises a shape memory element, and wherein, when selectively energized, the shape memory element is configured to change shape and thereby transmit a desired motion to the sensor.
47. The system of example 46 wherein the actuation element is composed of nitinol.
48. The system of example 46 wherein the actuation element is composed of a shape memory polymer.
49. The system of example 46 wherein the actuation element is composed of a pH-based shape memory material.
50. The system of example 44 wherein the actuation element comprises a bi-metallic strip.
51. The system of example 44 wherein the actuation element comprises a piezo-electric bimorph.
52. The system of example 44 wherein the actuation element is mechanically linked to the sensor.
53. The system of example 44, further comprising a power management system implanted in the patient and configured to provide power to at least one of the shunting element, sensor, and actuation element.
54. The system of example 44, further comprising a power management system implanted in the patient and configured to provide power to the actuation element, and wherein the power management system is out of electrical communication with the shunting element and the sensor.
55. The system of example 54 wherein the power management system comprises a power receiver operably coupled to an energy storage component, a switch, and a controller operably coupled between the switch and the actuation element.
56. The system of example 55 wherein the power receiver comprises a piezoelectric element configured to harvest energy from intentional or incidental bodily motion of the patient, pulsatile cardiac tissue motion, pulsatile hydraulic pressure variation in blood, and/or pulsatile Venturi effect pressure variations due to varying blood flow through the lumen.
57. The system of example 55 wherein the power receiver is configured to receive electromagnetic energy from ambient fields comprising one or more of the following: a radio receiver circuit capturing energy from cellular communications, WiFi, Bluetooth, WLAN (wireless local area network), WPAN (wireless personal area network), and WBAN (wireless body area network).
58. The system of example 44 wherein the sensor comprises a first sensor and the actuation element comprises a first actuation element, and wherein the system further comprises a plurality of second sensors and a plurality of second actuation elements coupled to corresponding second sensors, and further wherein the second actuation elements are configured to be selectively energized and transmit desired motion to the second sensors to limit or prevent tissue overgrowth and adhesion on at least a portion of the sensors.
59. A system for shunting blood between a left atrium and a right atrium of a patient, the system comprising:
   a shunting element having a lumen extending therethrough, wherein the lumen is configured to fluidly couple the left atrium and the right atrium when the shunting element is implanted in the patient;
   an implantable sensor operably coupled to the shunting element, wherein the sensor comprises a capacitive pressure sensor having a capacitor plate configured to deform under pressure,
   wherein, after implantation within the patient, the capacitor plate is adapted to (a) transduce pressure information and (b) convert received electrical energy to motion for periodic actuation of the sensor itself to limit or prevent tissue overgrowth and adhesion on at least a portion of the sensor.
60. The system of example 59, further comprising a power management system implanted in the patient and configured to provide power to at least one of the shunting element and the implantable sensor.
61. The system of example 59, further comprising a power management system implanted in the patient and configured to provide power to the implantable sensor, and wherein the power management system is out of electrical communication with the shunting element.
62. A sensor apparatus configured to be implanted in a subject, the sensor apparatus comprising:
   at least one sensor; and
   at least one changeable member,
   wherein the at least one changeable member is configured such that, after being positioned at a target anatomical site within the subject, a position and/or a configuration of the at least one changeable member can be periodically adjusted to limit or prevent tissue overgrowth and adhesion on at least a portion of the at least one sensor.
63. The sensor apparatus of example 62 wherein the at least one changeable member is in operational communication with an actuator, and wherein the actuator is configured to be automatically actuated via a controller configured to be implanted in the subject.

### Conclusion

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a LA of a heart) and a second region (e.g., a RA or coronary sinus of the heart) of a patient. Although described in terms of a shunt between the atria, namely the left and right atria, one will appreciate that the technology may be applied equally to devices positioned between other chambers and passages of the heart, or between other parts of the cardiovascular system. For example, any of the shunts described herein, including those referred to as “interatrial,” may be nevertheless used and/or modified to shunt between the LA and the coronary sinus, or between the right pulmonary vein and the superior vena cava. Moreover, while the disclosure herein primarily describes shunting blood from the LA to the RA, the present technology can be readily adapted to shunt blood from the RA to the LA to treat certain conditions, such as pulmonary hypertension. For example, mirror images of embodiments used to shunt blood from the LA to the RA can be used to shunt blood from the RA to the LA.

As described above, embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna that is capable of receiving and/or reading an externally-provided signal which may be used to power the device, charge a battery, initiate a reading from a sensor, or for other purposes. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe apparatuses implanted within certain parts of the body, it should be appreciated that similar embodiments could be utilized for apparatuses implanted in or positioned at a variety of other other regions of the body.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

What is claimed is:

1. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient;
a sensor configured to be implanted in the patient and operably coupled to the shunting element, wherein the sensor is configured to measure one or more parameters of the system and/or one or more physiological parameters of the patient; and
an actuation element coupled to the sensor, wherein the actuation element is configured to be selectively energized and reposition the sensor to reduce the likelihood of tissue ingrowth/overgrowth and adhesion on at least a portion of the sensor.

2. The system of claim 1, further comprising a battery configured to be implanted in the patient and store energy, wherein the energy stored in the battery can be used to actuate the actuation element.

3. The system of claim 1 wherein the actuation element comprises a shape memory element, and wherein, when selectively energized, the shape memory element is configured to change shape and thereby transmit a desired motion to the sensor.

4. The system of claim 3 wherein the actuation element is composed of nitinol.

5. The system of claim 3 wherein the actuation element is composed of a shape memory polymer.

6. The system of claim 3 wherein the actuation element is composed of a pH-based shape memory material.

7. The system of claim 1 wherein the actuation element comprises a bi-metallic strip.

8. The system of claim 1 wherein the actuation element comprises a piezo-electric bimorph.

9. The system of claim 1 wherein the actuation element is mechanically linked to the sensor.

10. The system of claim 1, further comprising a power management system configured to be implanted in the patient and provide power to at least one of the shunting element, sensor, and actuation element.

11. The system of claim 1, further comprising a power management system configured to be implanted in the patient and provide power to the actuation element, and wherein the power management system is out of electrical communication with the shunting element and the sensor.

12. The system of claim 11 wherein the power management system comprises a power receiver operably coupled to an energy storage component, a switch, and a controller operably coupled between the switch and the actuation element.

13. The system of claim 12 wherein the power receiver comprises a piezoelectric element configured to harvest energy from intentional or incidental bodily motion of the patient, pulsatile cardiac tissue motion, pulsatile hydraulic pressure variation in blood, and/or pulsatile Venturi effect pressure variations due to varying blood flow through the lumen.

14. The system of claim 12 wherein the power receiver is configured to receive electromagnetic energy from ambient fields comprising one or more of the following: a radio receiver circuit capturing energy from cellular communications, WiFi, Bluetooth, WLAN (wireless local area network), WPAN (wireless personal area network), and WBAN (wireless body area network).

15. The system of claim 1 wherein the sensor comprises a first sensor and the actuation element comprises a first actuation element, and wherein the system further comprises a plurality of second sensors and a plurality of second actuation elements coupled to corresponding second sensors, and further wherein the second actuation elements are configured to be selectively energized and transmit desired motion to the second sensors to limit or prevent tissue overgrowth and adhesion on at least a portion of the sensors.
